(19) United States
(12) Patent Application Publication
(10) Pub. No.: US 2015/0306330 A1
(43) Pub. Date: Oct. 29, 2015

(54) Mask Selection System

(71) Applicant: MaskSelect, Inc., San Diego, CA (US)

(72) Inventor: Ron Richard, San Diego, CA (US)

(21) Appl. No.: 14/700,109

(22) Filed: Apr. 29, 2015

Related U.S. Application Data
(60) Provisional application No. 61/985,463, filed on Apr. 29, 2014.

(51) Int. Cl. A61M 16/06; G06T 7/00; G06K 9/00; H04N 7/18

(52) U.S. Cl. CPC A61M 16/0605; H04N 7/183; G06T 7/0002; G06K 9/00221; G06T 2207/30201; A61M 2016/0661; A61M 2205/3306; A61M 2205/50

(57) ABSTRACT

Methods and systems, including computer program products, are provided for selecting a mask that is worn by a subject. A system can include an application that allows a subject to input information concerning the subject, such as sleeping habits. The subject then takes a photo of his or her face to help select the mask. The system then uses the collected data, comparing it to a database of known masks, to display results of masks that are most likely to fit the subject and his or her needs.

### CROSS-REFERENCES TO RELATED APPLICATIONS

This Application claims priority from U.S. Provisional Patent Application No. 61/985,463, filed Apr. 29, 2014, which is hereby incorporated by reference in its entirety as if fully set forth herein.

### FIELD

The subject matter described herein relates to selecting a mask for a subject, particularly a mask to treat sleep apnea.

### BACKGROUND

Sleep apnea has become a major problem facing individuals. The diagnosis and treatment of obstructive sleep apnea continues to grow at a rate of over 8% per year based in industry reports and will remain at that rate in the US for the next 3-5 years. Due to awareness related to the signs and symptoms of sleep apnea associated with several chronic diseases such as hypertension and diabetes and greater percentage of these users are now being screened and tested for obstructive sleep apnea, which has resulted in increased numbers of users receiving CPAP (“Continuous Positive Airway Pressure”) therapy as the leading option after diagnosis. It is estimated that in the US market, current daily users of CPAP therapy now exceeds 6 million and on average users refresh their supplies (CPAP masks and accessories) used in their treatment 2-3 times per year. A key component in the treatment of CPAP users is getting the user compliant short/long term. Compliance with CPAP therapy is closely aligned with comfort in wearing and using the therapy. Thus, there is a need for an efficient and easy system and method to make sure the mask an individual uses is properly selected.

### SUMMARY

Methods and systems, including computer program products, are provided for selecting a mask to be worn by a subject. The subject can be the user of the system, or the user can be a second person that uses the system and inputs information and data concerning the subject. For some example implementations, a system can include an interactive platform that includes a mask database, user provided input, a photograph of the subject, a sizing mechanism, and an algorithm to determine the best choices for a mask for this particular subject. A method can include acceptance of personal information regarding the subject, taking a photo of the subject's face, sizing a mask around the subject's face, matching criteria that retrieves data from a database of masks, and outputting the mask choices to a subject.

Provided in some embodiments is a method that includes executing an application on a mobile device having a camera and a display; receiving data representing at least one characteristic of a subject; receiving an image of at least a portion of a face of the subject from the camera; determining one or more measurements of the face based on the image; determining, based on the one or more measurements, at least one mask that is suitable for at least a portion of the face of the subject; receiving an input that selects one of the at least one suitable mask; and displaying on the display an output of the selected mask.

Provided in another embodiment is a system for selecting a mask comprising: a database storing information related to one or more masks; a smartphone associated with the database, the smartphone comprising a camera, a display, and a processor; and an executable application for execution by the processor, the application being configured to: control the camera to receive an image of at least a portion of a face of a subject; determine one or more measurements of the at least the portion of the face of the subject based on the image; access information for at least one mask from the database based on the one or more measurements; generate a representation of the information on the display; and receive a selection of one of the a least one masks.

Provided in another embodiment is a method comprising: obtaining a unique code; downloading an application onto a mobile device, the mobile device having a camera and a display; entering the unique code in the application; executing the application; receiving data representing at least one characteristics of a subject; receiving a single image of at least a portion of a face of the subject from the camera; determining information from the image, wherein said information comprises pupillary distance; comparing the information to data relating to one or more masks; determining, based on the comparison of the information to the data relating to one or more masks, at least one suitable mask; displaying on the display an output of the at least one suitable mask; and receiving an input that selects at least one of the suitable masks.

Aspects and features of the invention may be implemented in systems, apparatus, methods, and/or articles depending on the desired configuration. The details of one or more variations of the subject matter described herein are set forth in the accompanying drawings, appendixes and the description below. Features and advantages of the subject matter described herein will be apparent from the description, appendixes and drawings, and from the claims.

### BRIEF DESCRIPTION OF THE FIGURES

The present invention is illustrated by way of example, and not by way of limitation, in the figures of the accompanying drawings in which:

FIGS. 1A and 1B show a flow chart disclosing steps used in an embodiment of the present invention.

FIG. 2 shows an embodiment of the present invention as displayed on a smartphone.

FIG. 3 shows an embodiment of the present invention as displayed on a smartphone.

FIG. 4 shows an embodiment of the present invention as displayed on a smartphone.

FIG. 5 shows an embodiment of the present invention as displayed on a smartphone.

FIG. 6 shows an embodiment of the present invention as displayed on a smartphone.

FIG. 7 shows an embodiment of the present invention as displayed on a smartphone.

FIG. 8 shows an embodiment of the present invention as displayed on a smartphone.

FIG. 9 shows an embodiment of the present invention as displayed on a smartphone.

FIG. 10 shows a database used according to an embodiment of the present invention.

### DETAILED DESCRIPTION OF THE DISCLOSURE

The following description and the drawings illustrate specific embodiments sufficient to enable those skilled in the art to practice the system and method described. Other embodiments may incorporate structural, logical, process and other changes. Examples merely typify possible variations. Individual components and functions are generally optional unless explicitly required, and the sequence of operations may vary. Portions and features of some embodiments may be included in or substituted for those of others.

Traditional methods of selecting a mask for a subject can be frustrating, inefficient, and tedious. At times, multiple attempts are made whereby a subject tries on various masks, but is unable to actually determine if a mask is the right size, fit and comfort for that particular subject. A system that allows a subject to automatically determine which mask is likely to be a good fit is needed to save the subject time, money and effort investing in masks that are not comfortable or do not fit.

A CPAP system can consists of a device that provides airflow and in doing so generates a positive pressure when connected to a tube, which in turn is attached to a mask interface. The mask is helpful to the success of the therapy in many ways. It creates the seal required to generate and sustain positive pressure levels, which is associated with the Rx from the physician, derived during the overnight sleep study. The mask also has to be comfortable enough to wear for several hours while the subject is sleeping. Poor fitting masks can cause skin breakdown and if they do not fit well can leak and produce sub-therapy like results.

An embodiment of the present invention provides an easy to use website or app (or application) that can assist the provider or physician and subject in making current or future mask selections that best match their facial profiles.

The process of setting a subject up on CPAP therapy varies widely from provider to provider and usually takes about 1 hour for a trained mask provider to setup the device and educate the subject on a system consisting of a CPAP blower, tubing, humidifier and mask. The mask is often chosen by the technician and provided to the subject with other components. The device and mask are typically covered under most insurance programs. The mask can be replaced on varying frequencies depending on the subject's coverage plans. But often times subjects pay for masks out of pocket and use several different types of mask interfaces due to comfort and fitting issues. Alternatively, changes that occur over time such as gaining or losing weight, needing dentures or dental appliances, growing a beard or moustache, changing hairstyles and many other factors, can affect the subject's fit with their CPAP mask. If the mask does not fit properly, is old or worn, or is just not right for the subject, it can leak air around the eyes and face which causes the subject to wake up and lose sleep. This is why getting the right mask fit is important for short as well as long term compliance. If the subject gets frustrated with annoying issues like mask leak or poor fitting masks, it can very quickly cause pressure sores on the bridge of the nose, the subject will stop using therapy and become sleep deprived, which can result in their symptoms returning.

Another issue is that over time or after an initial setup, a subject's facial profiles change, such as based on the factors mentioned above. The subject may again struggle with trying to make a mask fit that seemed to work fine when they first started using their CPAP therapy but discover after growing a beard the mask doesn't fit anymore and leaks.

Under an embodiment of the present system, a subject can easily take a photo of their face and then run that image through the mask database allowing them to see masks that can better fit their current needs, thus eliminating leaks and improving compliance with their therapy—this can be done using the present system without having to visit a doctor or provider. The image is taken by the subject and a message is sent to their provider or physician showing what mask the application selected based on the photo and other screening protocols built into the app itself.

There are three or four commonly used mask interfaces on the market, which include: nasal masks; nasal pillows or prongs; full face masks; and hybrid masks.

The most popular style masks are nasal masks or nasal pillows. The top manufacturers of masks in the sleep space are ResMed, Philips/Respironics, Fisher Paykel and Innomed.

The app of the system can be in either iOS or Android formats (or any other computer or electronic format that is suitable to be used on a smartphone or computer) that are downloaded on a subject's smartphone or computer.

As used herein, a smartphone or computer can include, but is not limited to, an iPhone, Android phone, Windows phone, Blackberry, mobile phone, tablet, PC, computer, any other portable computing device, and the like.

The subject can be given the app by their home care provider at no charge or the subject can purchase it online. The subject can receive instructions as to how to activate and use the app by registering their information, how to take a picture of their face with their smartphone or computer, answering the filtering questions, and then sending the recommended mask via email to their provider or physician. The app can be downloaded to the subject's smartphone or computer and issued a unique ID code. In one example, the app can be used on a one-to-one basis, such that each unique ID code can be used for one subject and associated with a provider/physician. Alternatively, the ID code can be associated with a particular provider/physician, with that same code given out to each (or a subset) of their patients.

The app, when activated can appear on the subject's smartphone or computer. A guide can step the subject through a series of steps in order to successfully use the application such as centering the subject's face inside a visual outline. When the face is located within the frame, the app will prompt the subject to take a photo. The photo can be of the entire face of the subject or just a portion of the face of the subject.

According to one embodiment, the picture can capture from 1 to 5 key vector points on the face associated with the geometry of pre-selected masks manufactured by leading sleep companies, which are loaded, into the mask database. These vector points can include the bridge of the nose, the distance between the nares, the distance between the bridge of the nose and the space below the nostrils, the distance between the bridge of the nose and the space below the lower lip, and the like. All of these vector points factored in with the distance between the two pupils can be used as measuring points on the photo. Upon taking the photo, using the pupil distance as a reference point, the system makes one or more measurements from the various vector points. These measurements are then compared to the measurements for the various masks in the database.

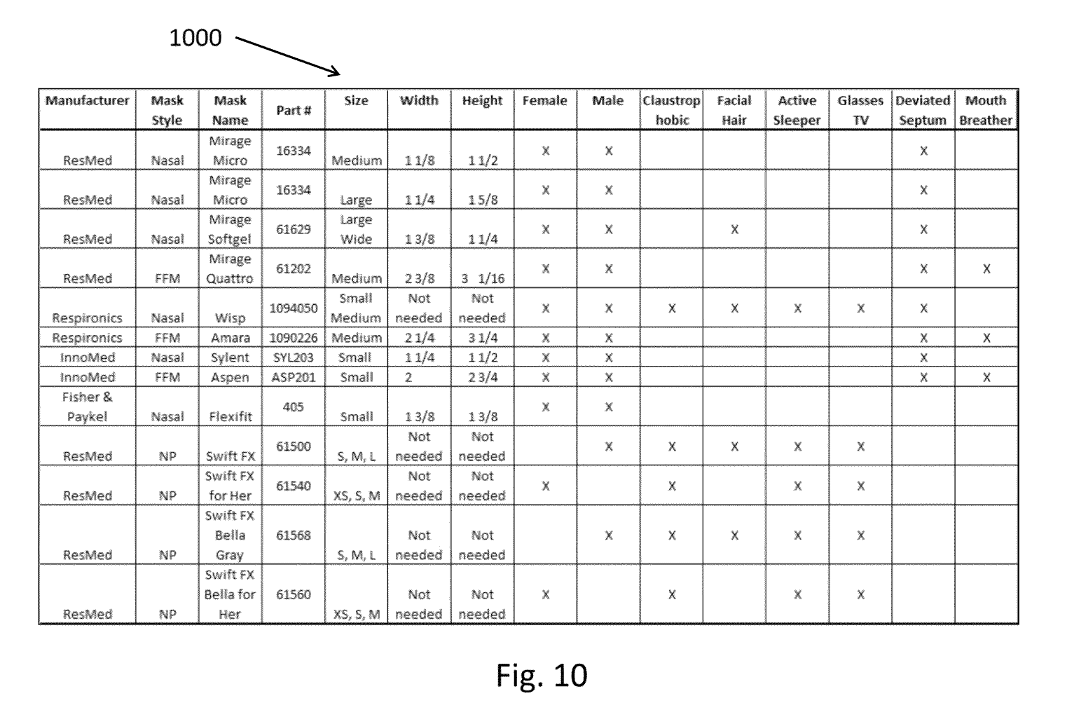

For example, as shown in FIG. 10, a database according to an embodiment of the present invention is shown. Database 1000 includes the manufacture's name, the mask style, the mask name, the part number, the size, the width, the height, whether it is for a male or female, whether it is for a claustrophobic subject, whether it is for a subject with facial hair, wither it is for an active sleeper, whether it is for a subject that wears glasses and watches TV at night, whether it is for a subject with a deviated septum, and whether it is for a mouth breather. Alternatively, other columns and input can be included in the database that would be relevant to the decision as to whether a particular mask is a good selection for a particular subject. The system can use this database to help narrow down the selected mask to one that is right for the subject. An example database is shown in FIG. 10.

The measurements for the various vector points can be used to compare to the database to determine the proper mask. For example, the pupillary distance can be used as a reference point. That allows a single two-dimensional image to be used to make the facial measurements. This also can replace the need to take a three-dimensional image or replace having to use a frontal and profile image. By knowing the pupillary distance, the vector points can be obtained using geometry and math. For example, if the pupil distance is known, then all the vectors points can be determined based on that single measurement, because the pupil distance serves as a reference point for the other measurements. When the vector points and measurements are calculated, the measurements are compared to the known measurements for each of the masks in the database.

By using these measurements, one or masks that actually fit the subject, based on the calculated measurements and the known sizes/tolerances of the masks, can be selected for the subject. Accordingly, mask(s) with the measurements that best fit the measurements of the subject taken from the photo are the mask(s) the system can select.

Alternatively, the mask can be sized around the nose and mouth, based on the photo. The recognition engine reviews the photo of the subject's face and runs this through the pre-loaded database of masks consisting of nasal, full face, pillows and hybrids. In one embodiment, the pupil distance is automatically calculated, for example by the camera knowing the distance the subject's face is from the camera—if that distance is known then the pupil distance can be measured using that known distance.

Once the picture is taken, the subject can receive a message from the app giving the subject the top three (or some other number, such as top one, top two, top four, top five, or the like) suggested mask interfaces that best match the subject's facial profile and based on the answers given to the filtering questions. If there is no match found, the app can instruct the subject to retake the photo. If no match is found again, the subject can contact the system (via phone, email, SMS, direct chat, or the like) and receive additional instructions to get a better image or physically measure the distance between their pupils and manually enter that number then re-take the photo. If again, no match is found the subject can be instructed to make an appointment to visit his or her provider or physician.

The database can consist of 50-75 (or more or less) of the most popular masks used on the market and be updated regularly, such as every 3-4 months. Subjects and providers who have used the app can be sent an email suggesting they download the new app or a message can appear on their smartphone or computer prompting them to upload the most current version of the application, such that the database on the smartphone is the most current database.

When a subject is issued the app via a home care provider or a physician, an email can be imbedded in the app that the provider selects to give instructions to the subject from the provider or physician on how the subject can utilize the application. Providers or physicians can purchase as many apps (or ID codes) as they wish and use them on future subjects. Alternatively, providers or physicians can contact existing subjects and offer them an invite or a link or information on how to download the app as a means of staying connected with their subjects and assisting with future mask orders or problems, for example, giving each subject a unique ID code.

If a subject wishes to purchase the application they can do so by selecting and downloading it from an app store such as the Apple store, Amazon, Google Play store, or the like, and use their own unique ID code to activate the app, using it as many times as they wish. The subject can send the information to a provider's email if they wish to share it, or simply go online and purchase the masks they have selected using the application.

Once a subject downloads the app, and certain mask(s) are selected, the subject can send an email to the provider, which activates the app to order one or more of the masks the app has selected. The activation codes can be linked to the provider, linking the subject and provider together for ongoing communications related to the mask selections. Alternatively, the subject can directly purchase the selected mask from an online mask store or an order can automatically be placed for the subject (such as through a provider or directly through a retailer).

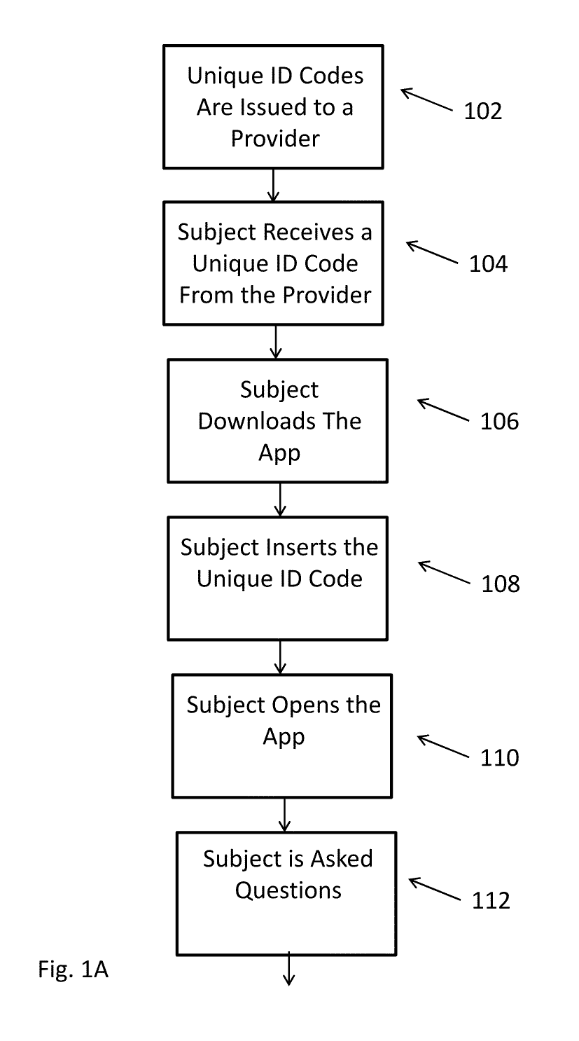

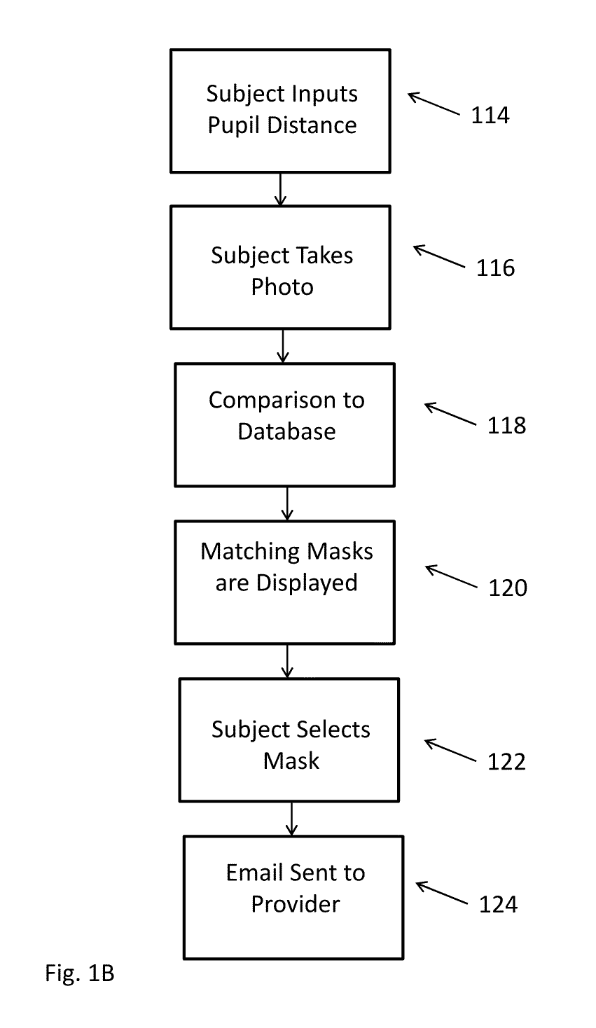

According to one embodiment, the steps can include the following, as shown in FIG. 1A-1B. First, at 102, unique ID codes are issued to a provider. Second, at 104, the subject receives a unique ID code from the provider. Third, at 106, the subject downloads the app. Fourth, at 108, the subject inserts the unique ID code on the display or within the application, linking the subject with the provider in the app. Fifth, at 110, the subject opens the app. Sixth, at 112, the subject is then asked a series of questions to help the app pick the appropriate mask. A subject can select boxes next to the options that are applicable, select yes or no for each question, or the like. These questions can include, but are not limited to:

“Are you a male or female?” Mask fitting can be affected by whether the subject is a male or female.

“Are you claustrophobic?” Many patients suffer from claustrophobia and find it uncomfortable to wear something that covers their face. There are interfaces that are better options for patients with claustrophobia and it can take time to find the right mask.

“Do you have a beard, mustache, goatee or any facial hair?” preferably for male subjects. Patients with beards and moustaches can experience mask leaks due to facial hair. If the patient is initially using a mask and did not have facial hair then grows a beard or moustache, the patient may need a different type of mask to reduce leaks.

“Are you an active sleeper?” If a patient sleeps on their side or stomach there are certain mask designs that can accommodate movement during sleep better than others.

“Do you read or watch television in bed?” If the patient wants to read or watch TV while using their CPAP system before falling asleep, there are masks designed to allow the subject to accomplish that goal.

“Do you wear glasses?” If the patient wears glasses, there are masks designed to allow the subject to wear glasses.

“Do you have weak hands?” If a patient has difficulty or dexterity issues with their hands, there are specific masks and headgear that are easier to work with than others are in terms of putting them on or removing them.

“Do you have a big round or wide face that requires a larger CPAP headgear?” If a patient has a big, round or wide face, there are masks specially designed for these individuals.

“Are you a side sleeper?” There are several options for patients that are active sleepers and prefer sleeping in different positions such as their sides.

“Are you a stomach sleeper?” There are a few masks available that work well for patients that sleep on their stomachs and designed for active sleepers or stomach sleepers.

“Do you have a deviated septum?” Patients who have a deviated septum might be best suited wearing a full-face mask or hybrid mask that utilizes nasal pillows and oral cushions.

Seventh, at 114, after answering the various questions (or before), the subject can input the pupil distance, or the distance between the subject's pupils. By inputting the pupil distance, the app has an anchor to make measurements. The known pupil distance allows the app to see the two pupils and know all other measurements related to the photo of the subject's face. Alternatively, the pupil distance can be automatically calculated by the application using other known measurements, such as the distance the camera is from the face of the subject.

The typical adult male has a pupil distance of between 2.16 to 2.75 inches. An example of a typical distance for a male is 2.46 inches. The typical adult female has a pupil distance of between 2.09 to 2.48 inches. An example of a typical distance for a female is 2.32 inches. The subject can either input their own pupil distance, or use the known average for male or femles. Alternatively, the system can automatically measure the subject's pupil distance.

Eighth, at 116, the subject takes the photo, such as by lining up his or her face in a specified box or circle. Once the subject's photo is taken, a drawn out “mask” can appear that can be sized over the subject's nose and mouth, simulating the mask. Alternatively, points can be moved and aligned on the subject's photographed face, to demonstrate known markers to select the mask, such as the above vector points.

Ninth, at 118, after the subject is satisfied with the photo and the drawn out mask/markers, the system then uses the data and accesses its known list of masks. Based on all the data provided, the system determines which masks are best for this particular subject. For example, the system can calculate the measurements of the vector points noted above, by using the pupil distance as a reference. Alternatively, the system can just measure the vector points.

According to one embodiment, metrics used in calculating the appropriate mask include the vector points and the distance between the pupils along with the answers to the filtering questions answered by the patient. The image or photo is mapped to the cross sectional database of masks stored in the database that fit within the answers to the questions by the subject. Alternatively, the measurements on the subject's photo are compared the measurements of the known masks that fit within the questions answered by the subject. For example, a mask may have a measurement of 0.2-0.4 inches between the nares and 1.1-1.4 inches from the bridge of the nose to the space below the lower lip. If the subject's measurements fit within these measurements (such as 0.3 inches between the nares and 1.2 inches from the bridge of the nose to the space below the lower lip), this mask can be selected.

Tenth, at 120, the best-matched masks are then displayed for the patient to review and select. The information can be displayed as images or texts showing a predetermined number of selected masks (or all masks that may be a match). One reason to limit the results could be the provider associated with the ID could have a preference for certain brands of masks. The system could be used such that only those brands, that are also successful matches for the subject, are displayed to the subject.

Eleventh, at 122, after the results are displayed, the subject can click on the various results. By doing so, the subject can see additional data on the various mask, see further information about the masks, see reviews of the masks, or the like. This can help the subject make a decision based on the displayed results. In one embodiment, the display can include the measured results for the subject.

Twelve, at 124, after a decision has been made, an email or other notification can be sent to the provider such that the provider can order or provide to the subject the selected mask. Alternatively, an order can be automatically placed to purchase the chosen mask. Alternatively, the provider or physician does a further review and discusses the choices with the subject.

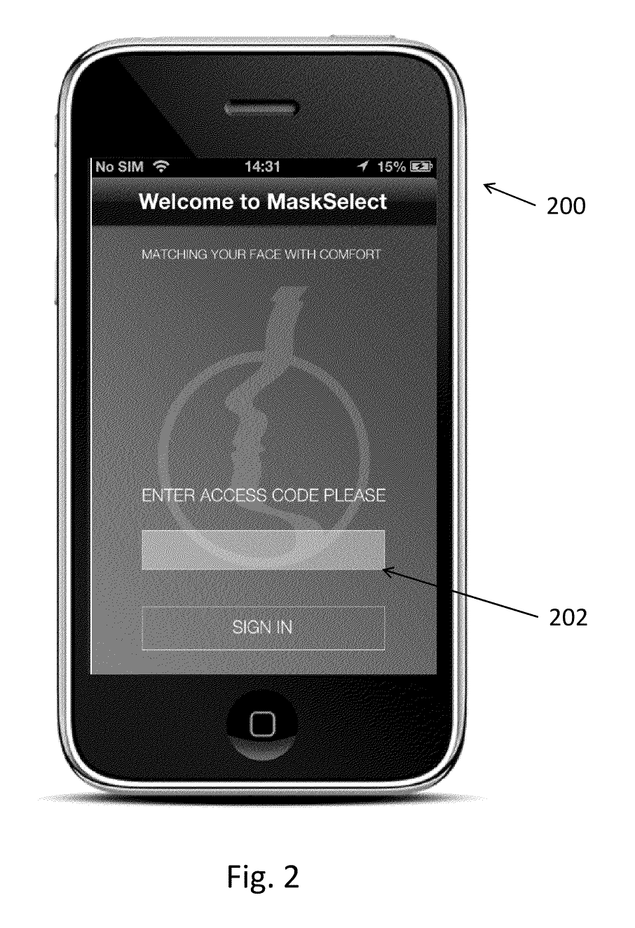

FIGS. 2 through FIGS. 9 show embodiments of the present invention as displayed on a smartphone.

FIG. 2, at 200, shows a welcome screen after the app has been downloaded and opened on a smartphone. Access code area 202 is where the access code can be entered.

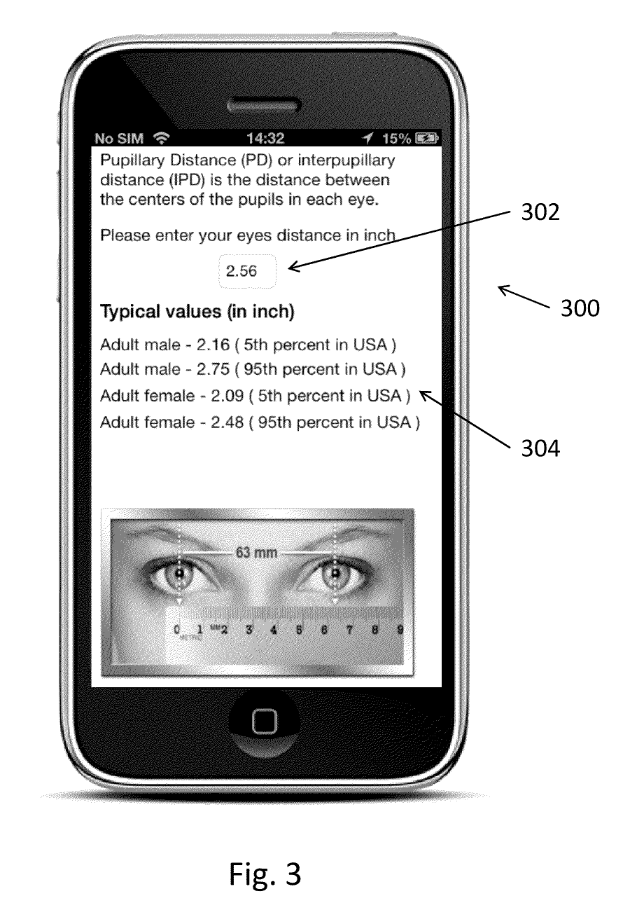

FIG. 3, at 300, shows a screen whereby a subject enters the pupillary distance—the distance between the centers of the pupils in each eye. The subject enters his or her pupillary distance at 302. 304 shows typical values (in inches) of pupillary distance in common males and females, showing that the top 95% of males are 2.75 inches and the top 95% of females are 2.48 inches, with the bottom 5% of males being 2.16 inches and the bottom 5% of females being 2.09 inches. That means most males have a pupil distance between 2.16 and 2.75 inches and most females have a pupil distance between 2.09 and 2.48 inches. This information helps the subject know where typical pupil distance measurements fall.

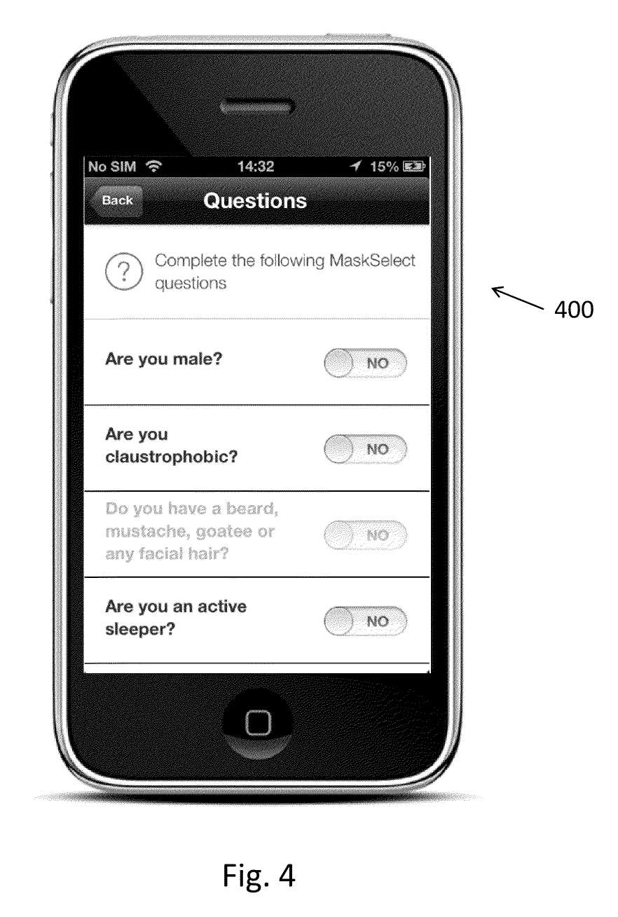

FIG. 4, at 400, shows questions a subject may be asked to help determine the mask that is best for him or her. For example, the questions can include “are you a male;” “are you claustrophobic;” “Do you have a beard, mustache, goatee or any facial hair;” “are you an active sleeper;” and the like.

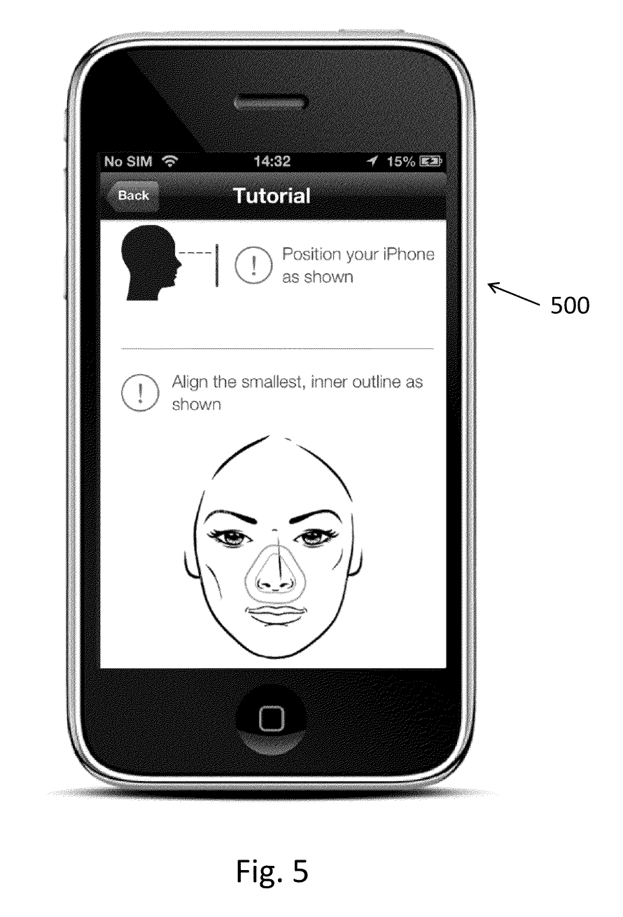

FIG. 5, at 500, shows a tutorial to help the subject take a photo of his or her face. The photo is taken using the camera on the smartphone.

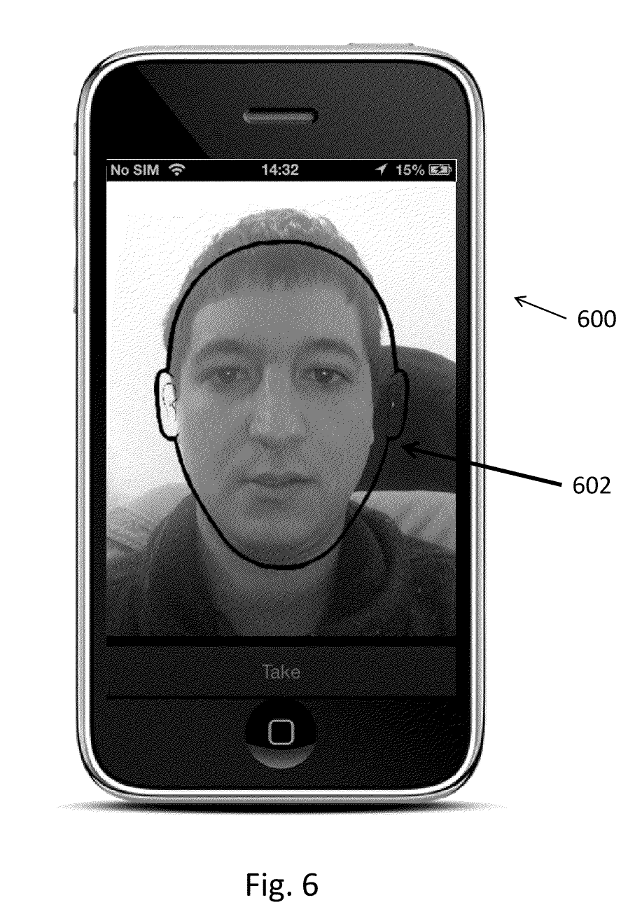

FIG. 6, at 600, shows a subject taking a picture of his or her face using his or her smartphone. The black guide 602, showing the outline of a head, can be used to help the subject position his or her face properly, within the proper distance and proportions. A single image of the subject's face is needed, as the vector points can be determined using this single photo. Alternatively multiple photos, and at multiple angles, may be taken and used to determine the proper measurements.

Once the photo is taken, the camera sends the image to the smartphone and is accessed within the app, allowing various measurements to be made. The pupillary distance is used as a reference guide to determine one or more measurements on the subject's face, such as the distance between the nares of the subject, the distance between the bridge of the nose and the space below the nostrils of the subject, the distance between the bridge of the nose and the space below the lower lip, and the like. The one or more measurements are then compared to the known suitable measurements for the masks within the database.

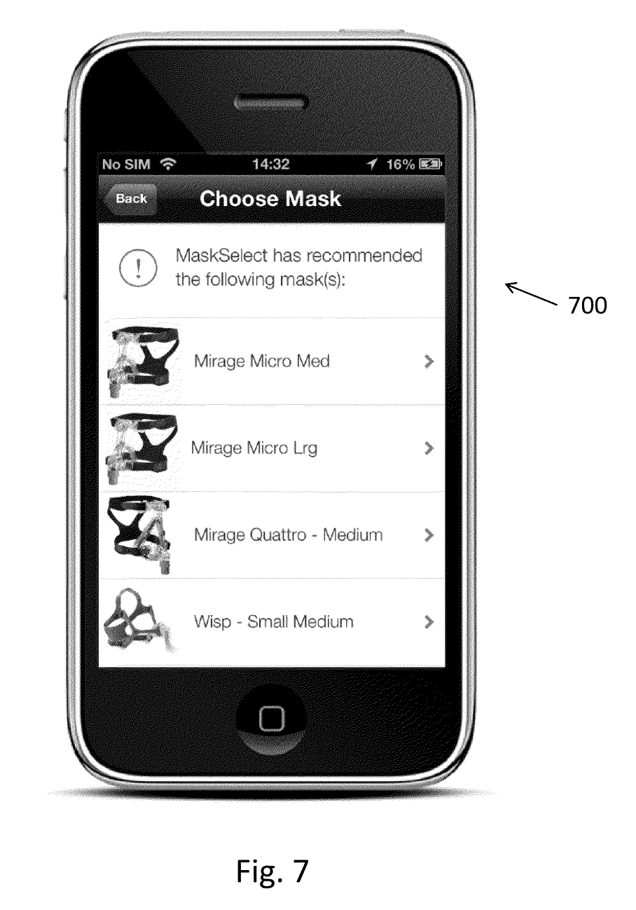

FIG. 7, at 700, shows the results of what masks the system recommends for the subject. The subjects selects which mask he or she desires.

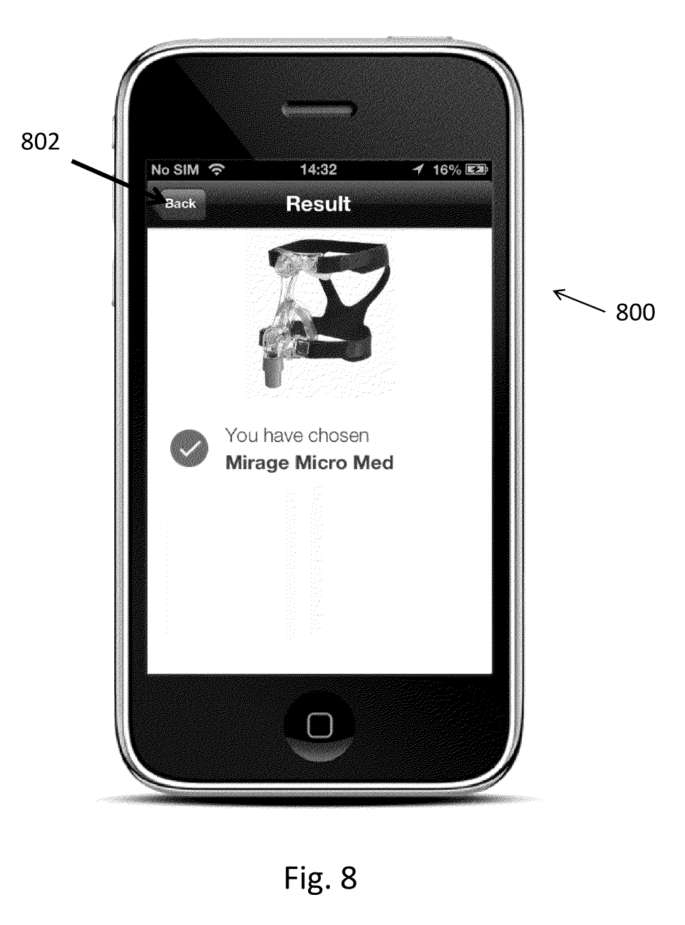

FIG. 8, at 800, shows the mask the subject selected. If the subject did not select this mask, he or she may select the back button 802, to return to result screen shown in FIG. 7 at 700.

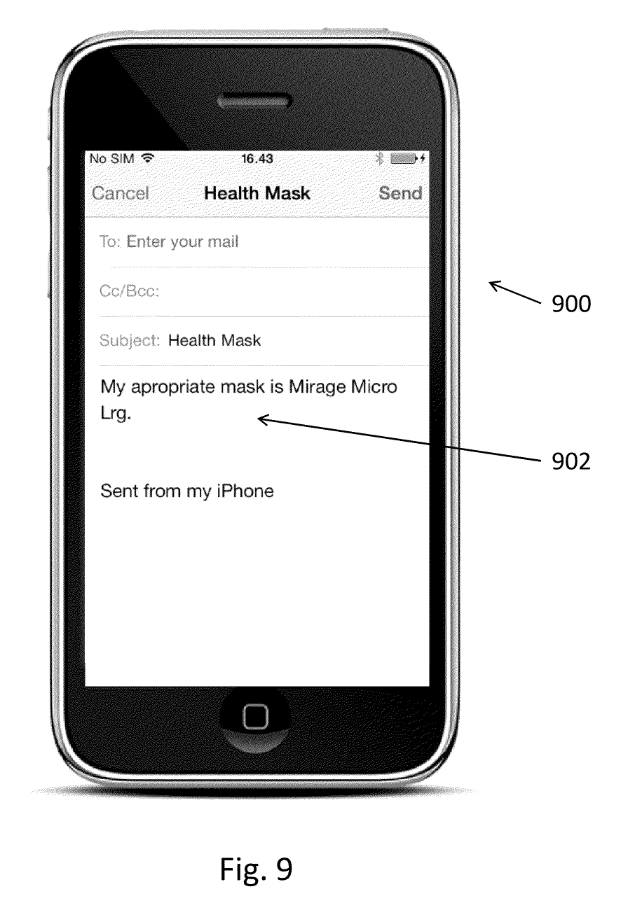

FIG. 9, at 900, shows an email that is sent to the provider, once the subject selects a mask. The email 902 informs the provider of the selection and allows the selection to be recorded. Alternatively, the mask can be ordered immediately via the system, such as if the subject's payment information has already been stored and can be accessed.

As an alternative embodiment, the app can also provide to the subject other accessories or items that may be useful to the subject. These can include suggestions based on the above information collected from the subject. For example, mattress, comforters, bedding, and other sleep accessories can be suggested to the subject based on the subject's previous input. Other sleep related products can be suggested as well, such as sleepwear accessories, pillows, sound masking devices, sleep comfort products, and the like. Advertising space can be included in the app, using advertisers, such as for example, pharmaceutical manufacturers, medical device manufacturers, retailers of sleep related products, and the like.

In an alternative embodiment, the app can be run using a kiosk for example one placed in a retail location such as a pharmacy, supermarket, or some location that provides masks. The subject can approach the kiosk and then interact with the app, including answering the above questions and having the kiosk take the picture of the subject, to help determine which mask(s) to recommend. The kiosk can be stationary or portable, such that it can be moved around the retail location. Alternatively, instead of a retail location, it can be in a doctor's office, sleep center, or any other location where subjects may be present that desire to know which mask to use.

As mentioned herein, the system herein can be presented to subjects, via a website or a dedicated application (e.g., app on a hand-held computing device), or via both a website and a dedicated application. The website and dedicated application can be used with desktop computers, laptop computers, mobile computing devices such as tablets, palm-sized devices (e.g., mobile phones), and wearable electronic interfaces (e.g., watches).

The subject matter described herein may be embodied in systems, apparatus, methods, and/or articles depending on the desired configuration. For example, application (or one or more components therein) and/or the processes described herein can be implemented using one or more of the following: a processor executing program code, an application-specific integrated circuit (ASIC), a digital signal processor (DSP), an embedded processor, a field programmable gate array (FPGA), and/or combinations thereof. These various implementations may include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device. These computer programs (also known as programs, software, software applications, applications, components, program code, or code) include machine instructions for a programmable processor, and may be implemented in a high-level procedural and/or object-oriented programming language, and/or in assembly/machine language. As used herein, the phrase “machine-readable medium” refers to any computer program product, computer-readable medium, apparatus and/or device (e.g., magnetic discs, optical disks, memory, Programmable Logic Devices (PLDs)) used to provide machine instructions and/or data to a programmable processor, including a machine-readable medium that receives machine instructions. Similarly, systems are also described herein that may include a processor and a memory coupled to the processor. The memory may include one or more programs that cause the processor to perform one or more of the operations described herein.

Because of the high-level nature and complexity of the selections and methods described herein, including the multiple and varied combinnations of different masks, the calculations, computations and selections cannot be done in real time quickly or at all by a human. The processes described herein rely on the machines described herein.

Although a few variations have been described in detail above, other modifications or additions are possible. In particular, further features and/or variations may be provided in addition to those set forth herein. For example, the implementations described above may be directed to various combinations and subcombinations of the disclosed features and/or combinations and subcombinations of several further features disclosed above. In addition, the logic flow depicted in the accompanying figures and/or described herein in a certain order does not require the particular order stated or shown, or sequential order, to achieve desirable results. In various example implementations, the methods (or processes) can be accomplished on mobile station/mobile device side or on the server side or in any shared way between server and user equipment/mobile device with actions being performed on both sides. The phrases “based on” and “based on at least” are used interchangeably herein. Other implementations may be within the scope of the following claims.

What is claimed is:

1. A method comprising:
executing an application on a mobile device having a camera and a display;
receiving data representing at least one characteristic of a subject;
receiving an image of at least a portion of a face of the subject from the camera;
determining one or more measurements of the face based on the image;
determining, based on the one or more measurements, at least one mask that is suitable for at least a portion of the face of the subject;
receiving an input that selects one of the at least one suitable masks; and
displaying on the display an output of the selected mask.

2. The method of claim 1, further comprising:
obtaining a unique code from a provider; and
inputting the code on the display to obtain the application.

3. The method of claim 1, wherein the image is a single, two-dimensional image.

4. The method of claim 1, wherein receiving data representing at least one characteristic of a subject comprises obtaining information concerning the subject by asking one or more questions about the subject and wherein the one or more questions are selected from the group consisting of: whether the subject is a male or female, whether the subject is claustrophobic, whether the subject has facial hair, wither the subject is an active sleeper, whether the subject wears glasses; whether the subject watches TV at night, whether the subject has a deviated septum, and whether the subject is a mouth breather.

5. The method of claim 1, wherein receiving data representing at least one characteristic of a subject comprises obtaining a pupil distance of the subject.

6. The method of claim 1, further comprising displaying a guide wherein the at least a portion of the face of the subject is to be fit within the guide.

7. The method of claim 1, wherein determining one or more measurements comprises using vector points from the image.

8. The method of claim 1, further comprising displaying one or more accessories related to the selected mask.

9. The method of claim 1, further comprising sending an email to a provider wherein the email comprises the identity of the selected mask.

10. A system for selecting a mask comprising:
a database storing information related to one or more masks;
a smartphone associated with the database, the smartphone comprising a camera, a display, and a processor; and
an executable application for execution by the processor, the application being configured to:
control the camera to receive an image of at least a portion of a face of a subject;
determine one or more measurements of the at least the portion of the face of the subject based on the image;
access information for at least one mask from the database based on the one or more measurements;
generate a representation of the information on the display; and
receive a selection of one of the a least one masks.

11. The system of claim 10, wherein the one or more measurements are computed using vector points from the image.

12. The system of claim 10, wherein the image is a single, two-dimensional image.

13. The system of claim 12, wherein the one or more measurements are determined using information provided by the subject.

14. The system of claim 12, wherein the one or more measurements comprise pupillary distance.

15. The system of claim 12, wherein the information for at least one mask from the database is further based on at least one characteristic of the subject.

16. The system of claim 15, wherein the at least one characteristic of the subject comprises whether the subject is claustrophobic or whether the subject has facial hair.

17. The system of claim 10, wherein the executable application for execution by the processor is further configured to send an email to a provider, wherein the email comprises the identity of the selected mask.

18. A method comprising:
obtaining a unique code;
downloading an application onto a mobile device, the mobile device having a camera and a display;
entering the unique code in the application;
executing the application;
receiving data representing at least one characteristics of a subject;
receiving a single image of at least a portion of a face of the subject from the camera;
determining information from the image, wherein said information comprises pupillary distance;
comparing the information to data relating to one or more masks;
determining, based on the comparison of the information to the data relating to one or more masks, at least one suitable mask;
displaying on the display an output of the at least one suitable mask; and
receiving an input that selects at least one of the suitable masks.

19. The method of claim 18, further comprising ordering the selected mask.

20. The method of claim 18, further comprising sending an email to a provider, wherein the email includes information concerning the selected mask.
